(19) United States
(12) Patent Application Publication
(10) Pub. No.: US 2021/0106074 A1
(43) Pub. Date: Apr. 15, 2021

(54) COUNTERBALANCE SYSTEM FOR WOMEN'S BRAS

(71) Applicant: Counterbalance Technology Inc., Southbury, CT (US)

(72) Inventor: John A. Bucciarelli, III, Southbury, CT (US)

(21) Appl. No.: 17/126,406

(22) Filed: Dec. 18, 2020

Related U.S. Application Data
(62) Division of application No. 15/360,225, filed on Nov. 23, 2016, now abandoned.
(60) Provisional application No. 62/259,317, filed on Nov. 24, 2015.

(51) Int. Cl. A41C 3/00

(52) U.S. Cl. CPC A41C 3/005; A61F 5/026; A41C 3/0035; A41C 3/0028

(57) ABSTRACT

Counterbalancing methods, systems and apparatus that balance breast weight load to a user's back. A body portion is supported along the user's back, and a plurality of pockets reside one the body portion. Elongated flexible connector straps extend up from the body portion, over the shoulders, and connect to a bra cup. One or more weights retained within the plurality of pockets pull the bra cup portion upward and lift up the breasts. The weight measure of these weights is substantially equivalent to a combined breast weight measure to equilibrate the breast weight to the combined weight measure across the user's body to correct for breast weight load imbalance at the user's front and improve posture.

This application is a Divisional of U.S. patent application Ser. No. 15/360,225 filed Nov. 23, 2016, which claim priority to U.S. Provisional Patent Application Ser. No. 62/259,317 filed Nov. 24, 2015.

### BACKGROUND OF THE INVENTION

### 1. Field of the Invention

The present invention is directed to a weighted counterbalancing system, and in particular, to systems and apparatus that counterbalance breast weight and correct posture.

### 2. Description of Related Art

There are a variety of different types of bras currently available in the market. A conventional bra is designed to at least include a pair of cup portions each receiving and retaining a breast. A bra may also include a pair of straps each extending from one of the cup portions up and over the wearer's shoulders, as well as a back portion that extends laterally from the cup portions around the wearer's rib cage and back. While the majority of bras are designed to secure and lift breasts in the cup portion, none of them take into consideration breast weight or the balance load of breasts on a person's posture.

In some currently available bra designs, breast weight is lifted and both loaded and pinned to the wearer's shoulders using a variety of different supports. Other known bras compress the breast weight to the chest (e.g., a sports bra). Women that wear these types of conventional bras, that either balance breast weight loads to the shoulders or compress breast weight to the chest, often experience considerable amounts of stress on the neck, shoulders, and/or back. All of this stress is a result of the ineffective breast support and breast weight load imbalance between the front and back of the woman's body. These stresses are undesirable as they may lead to bad posture, difficulty breathing, muscle and joint pain, neck pain, shoulder pain, and back pain.

The stresses and pain resulting from ineffective breast support and breast weight load imbalance may often be debilitating. For instance, for women with large breasts, the pain may be exacerbated due to the increased breast weight load at the front of the body leading to chronic pain. Chronic pain may cause headaches, fatigue, depression and irritability, resulting in a negative impact on one's physical capacity, functional capacity, quality of life, and overall feeling of well-being. Further as women age, their breasts begin to sag and shoulders naturally roll forward, which in turn puts compression in the thoracic region (i.e., the area where the ribs, shoulder blades and nerves come through a narrow triangle). This sagging and rolling forward of the shoulders, combined with changes in anatomic space in the female back, compress nerves to cause or increase pain even further.

While women may receive medications, physical therapy, surgery, or orthotic devices to address the pain and ailments resulting from breast weight load imbalance on the body, it would be advantageous to prevent and avoid these problems and complication from even occurring in the first instance.

### SUMMARY OF THE INVENTION

Bearing in mind the problems and deficiencies of the prior art, it is therefore an object of the present invention to provide systems and apparatus that counterbalance asymmetrical breast weight load imbalance between the front and back of a woman's body.

It is another object of the present invention to provide systems and apparatus that counterbalance breast weight across the body to improve and correct posture.

A further object of the invention is to provide systems and apparatus that counterbalance breast weight across the body to reduce or avoid stress on the neck, shoulders, and back.

It is yet another object of the present invention to provide systems and apparatus that counterbalance breast weight across the body to take pressure off the chest cavity, improve breathing, decrease muscle and joint pain, enhance a person's physical and functional capacities, improve quality of life, and provide an overall feeling of well-being.

Still other objects and advantages of the invention will in part be obvious and will in part be apparent from the specification.

The above and other objects, which will be apparent to those skilled in the art, are achieved in the present invention which is directed to a counterbalancing system for balancing breast weight load to a user's back. The system includes a body portion adapted to be supported along the user's back, and a plurality of pockets residing across the body portion at the user's back. Elongated flexible connector straps extend up from the body portion, up and over the user's shoulders, and connect to a cup portion that retains the user's breasts. One or more weights are retained within one or more of the plurality of pockets, thereby pulling the cup portion upward and lifting the user's breasts up. These weights have a combined weight measure substantially equivalent to a combined breast weight measure of the user's breasts, thereby equilibrating the breast weight and combined weight measure across the user' body to correct for breast weight load imbalance at the user's front.

In one or more embodiments, the body portion may be a component of a harness type system, a component of a bra having the cup portion, or a component of a top worn by the user. The system may further include a pair of lateral straps that wrap around the user's rib cage to secure the counterbalancing system to the user's body and/or a pair of shoulder pads adapted to be positioned on the user's shoulders. The connector straps may be substantially non-stretchable when connected to the body portion, and optionally may have fasteners connected to a distal end thereof for connection to the cup portion of the bra.

The present invention is further directed to a counterbalancing system for balancing breast weight load to a user's back. The system includes a body portion adapted to be supported along the user's back, and a plurality of pockets residing across the body portion. A pair of elongated connector straps extend upward from the body portion, up and over the user's shoulders, and connect to a cup portion of a bra that is retaining the user's breasts. A pair of lateral straps extend from the body portion and wrap around the user's rib cage to secure the body portion to the user's body. One or more weights are retained within one or more of the plurality of pockets residing on the body portion. These weights have a combined weight measure substantially equivalent to a combined breast weight measure of the user's breasts so that the weight(s) pull down on the pair of elongated connector straps, which in turn, pull up on the cup portion to lift the breast and correct for breast weight load imbalance at the user's front by equilibrating weight load at the user's back.

Still further, the present invention directed to a method for counterbalancing breast weight load to a user's back, the user wearing a bra having a cup portion retaining the user's breasts. The method includes: providing a body portion adapted to be supported along the user's back, the body portion having a plurality of pockets residing thereon; providing a plurality of weights; determining a breast weight of the breasts and a number of the plurality of weights that will have a weight load substantially equivalent to the breast weight; inserting one or more of the plurality of weights based on the determined number thereof into one or more of the plurality of pockets residing on the body portion; extending a pair of elongated connector straps connected to the body portion up and over the user's shoulders; connecting the pair of elongated connector straps to the cup portion of the bra retaining the user's breasts; and pulling up the cup portion to allow the body portion having the one or more weights residing in the one or more pockets to fall back behind the user's back; wrapping a pair of lateral straps extending from the body portion around the user's rib cage to secure the body portion to the user's body, whereby the one or more weights pull down on the pair of elongated connector straps which in turn pull up on the cup portion to lift the breast and correct for breast weight load imbalance at the user's front by equilibrating weight load at the user's back.

### BRIEF DESCRIPTION OF THE DRAWINGS

The features of the invention believed to be novel and the elements characteristic of the invention are set forth with particularity in the appended claims. The figures are for illustration purposes only and are not drawn to scale. The invention itself, however, both as to organization and method of operation, may best be understood by reference to the detailed description which follows taken in conjunction with the accompanying drawings in which:

FIG. 1A is a schematic first side plan view of a weighted counterbalancing apparatus and system in accordance with one or more embodiments of the invention;

FIG. 1B is a schematic second side plan view of the weighted counterbalancing apparatus and system of FIG. 1A;

FIGS. 2A-2D illustrate various views of a shoulder pad that may be used in the weighted counterbalancing system of the present invention;

FIGS. 3A-3B illustrate various views of weights used in combination with the weighted counterbalancing apparatus and system of the present invention;

FIG. 4 illustrates a weighted counterbalancing apparatus and system in accordance with one or more embodiments of the invention used in combination with a conventional bra;

FIGS. 5A-5D illustrate various views of the weighted counterbalancing apparatus of FIGS. 1A and 1B and system of FIGS. 2A-3B including shoulder pads and weights attached to a conventional bra worn by a woman;

FIGS. 6A-6D are various views of another weighted counterbalancing apparatus and system in accordance with the invention illustrating the benefits and results thereof;

FIG. 7 shows a chart of estimated breast weights for various bra sizes, and corresponding numbers of weights of FIGS. 3A-3B needed to counterbalance and equilibrate such breast weights; and

FIGS. 8-9 illustrate side views of different weighted counterbalancing apparatus and system embodiments in accordance with the invention.

### DESCRIPTION OF THE PREFERRED EMBODIMENT(S)

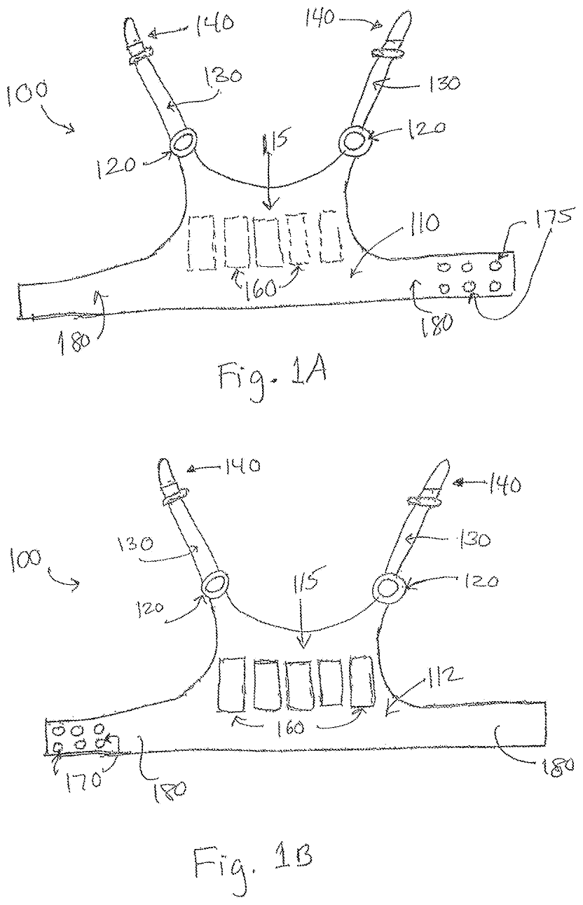

In describing the preferred embodiment of the present invention, reference will be made herein to FIGS. 1A-9 of the drawings in which like numerals refer to like features of the invention.

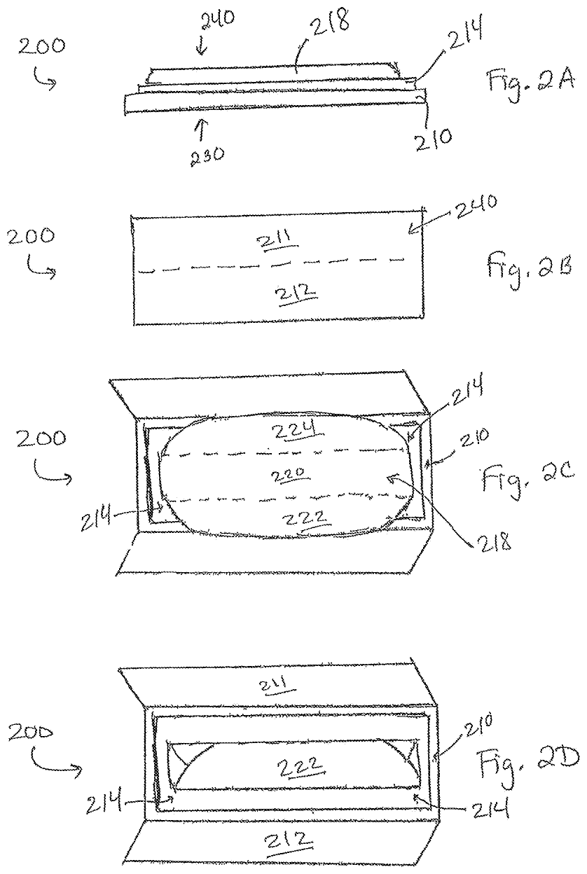

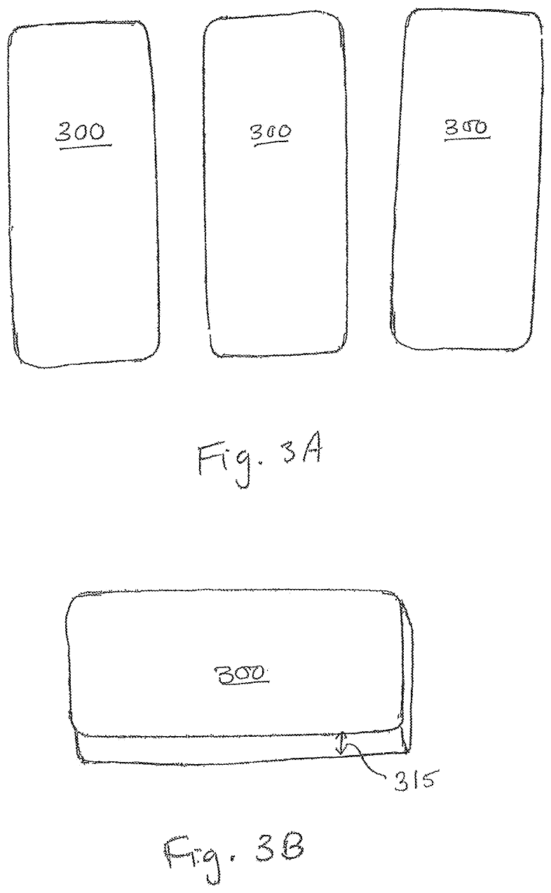

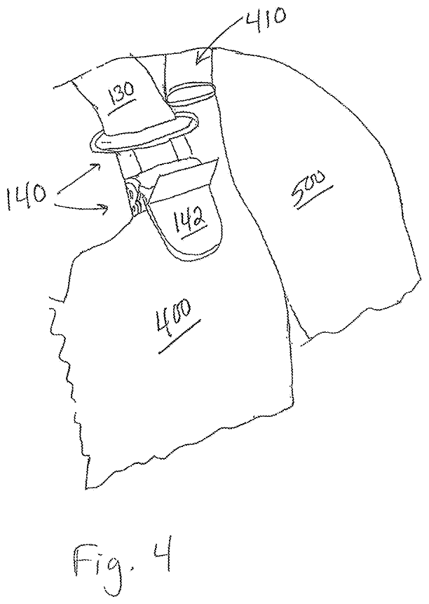

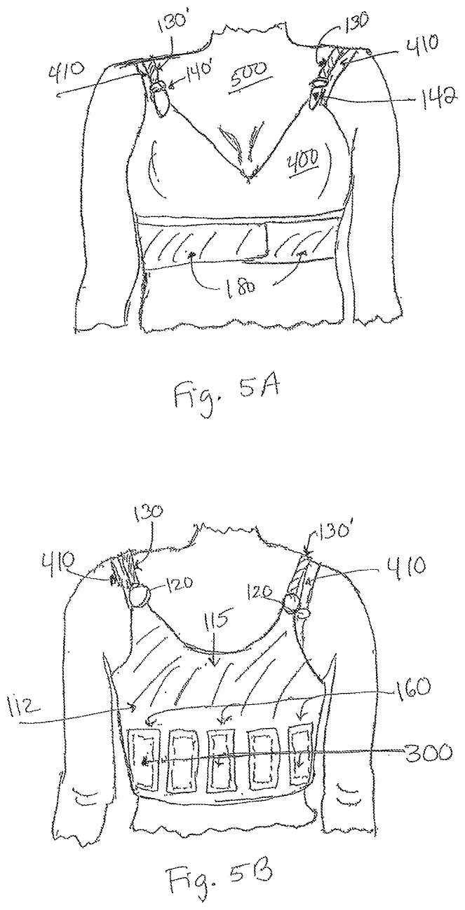

The present invention is directed to a weighted counterbalancing system, and in particular, to systems and apparatus that counterbalance breast weight and correct posture. As used herein, the term counterbalance refers to an equal distribution of weight, even balance, equilibrium, and/or to equal or offset weight. In one or more embodiments the apparatus and systems of the invention may be used in combination with a conventional bra 400. Referring to FIGS. 1A and 1B, in these embodiments the counterbalancing apparatus 100 resembles a harness and may include a body portion 115, connectors 120, a pair of straps 130, fasteners 140, first and second sides 110, 112, a plurality of pockets 160, a pair of lateral straps 180 and mating connection components 170, 175. The counterbalancing apparatus 100 may further include a pair of shoulder pads 200 as shown in FIGS. 2A-2D, and a plurality of weights 300 as shown in FIGS. 3A-3B.

In accordance with the invention, the counterbalancing apparatus 100 may include the body portion 115 connected to the pair of straps 130 via the connectors 120. These connectors 120 may include any type of connection means that allows the straps 130 to be securely attached to the body portion 115 of the apparatus. In certain embodiments, the straps 130 may be directly connected to the body portion 115 without the use of connectors 120. In further embodiments, the pair of straps 130 may be adjustable so as to adjust the straps of the apparatus 100 over a wearer's shoulders for secure attachment to a bra. The straps 130 may be composed of a non-stretch material that allows for counterbalancing the breast weight to the weights 300 at the back side of the user. For instance, the straps 130 may be composed of a non-stretch nylon material, cotton material or combination of nylon/cotton material.

As shown in FIG. 4, in one or more embodiments each strap 130 may be attached to a fastener 140 that includes a fastening portion 142 for attachment to a bra 400. The fasteners 140 may include any type of known fastening means that securely attaches to a conventional bra 400. In one or more embodiments, the fasteners 140 may include but are not limited to hooks, clips (e.g., pacifier clip or fabric clip), pins, clamps, alligator clamps, or any other type of fastening mean that may be removably attached to a fabric material.

Attached to the body portion 115 of the counterbalancing apparatus 100 is a plurality of pockets 160. The plurality of pockets 160 may be attached to the first side 110 or the second side 112 of the body portion. In one or more embodiments the plurality of pockets 160 are preferably attached to the second side 112 of the apparatus that does not contact the wearer's skin. It should be appreciated that in other embodiments the pockets 160 may be affixed to the first side 110. Depending on the size of the counterbalancing apparatus 100, the body portion 115 may be provided with any number of pockets 160 thereon. While the counterbalancing apparatus 100 may have an even number of pockets 160, in one embodiment it is preferred that the counterbalancing apparatus 100 has an odd number of pockets 160 to effectively counterbalance the weight load across the user's back. For instance, the body portion 115 may have anywhere from three (3) pockets 160 to five (5) pockets, or even more or less.

Each pocket 160 is securely fastened on at least three sides to the body portion 115 and has an open at a top portion thereof for receiving weights 300. The pockets 160 may be fastened to the body portion by any known fastening means including, but not limited to, stitching, glue, hook and loop (e.g., Velcro), and the like. Referring to FIG. 1A, stitching is used to securely attach the pockets 160 onto the apparatus 100 whereby such stitching is shown on the first side 110 of the body portion 115. In accordance with the invention, each of the plurality of pockets 160 has enough room to fit and hold at least one weight 300, and even two or more weights 300 in each pocket 160.

The body portion 115 of the counterbalancing apparatus 100 may be composed of a variety of different materials. In one or more embodiments the body portion 115 may be composed of a material that is able to slightly stretch to accommodate different user body shapes and types, and provide comfort to the user. In one or more preferred embodiments the body portion 115 may be composed of a rayon/spandex material, a brushed jersey material in a polyester, rayon and spandex blend, or other suitable sportswear type material. Preferably the material of the body portion 115 allows for stretch, comfort, moisture absorbency, and fabric breathability for moving body heat away from the user. The body portion 115 and overall apparatus 100 may be provided in a number of different sizes, materials, and fabric colors.

Extending from each lateral side of the body portion resides one of the lateral straps 180, which together, extend around opposing sides of the user's rib cage, and are securely fastened to each other at the front of the user's body. The lateral straps 180 may also be composed of a material that is able to slightly stretch to accommodate different user body shapes and types, and provide comfort to the user. Located at the opposing ends of each lateral strap 180 are a number of mating connection components 170, 175 that allow for adjustable sizing of the straps 180 around the user's rib cage. For instance, these mating connection components 170, 175 may be hook and loop fasteners, clips, hook-and-eyes closures, buttons, zippers, and another other fastening means that allows the straps to be adjustably secured to and around the user's rib cage or torso to accommodate user size.

Referring to FIGS. 2A-2D, the counterbalancing system of the invention may also include one or more removable shoulder pads 200, preferably a pair of shoulder pads 200, for use with the counterbalancing apparatus 100 to balance the weight load of the breasts and weights across the user's shoulders. The shoulder pads 200 may be provided in a variety of different sizes, and preferably are of a size sufficient to evenly distribute weight load across the shoulder area.

Each shoulder pad 200 may include one or more of the following layers: an outer cloth material 210, a foam pad 214, and/or a silicone pad 218. These layers may be removable from each other as well as adjustable for accommodating different sized shoulder pads and shoulder area. In one or more embodiments, the shoulder pads 200 may have a first surface 230 that contacts a user's skin, and a second surface 240 that wraps around and over the exposed portion of its respective strap 130 that it is secured to. The various layers of the shoulder pad 200 may each be used alone or in combination with one another. In those embodiments using a number of pad layers, each layer may be securely attached or affixed to one another, either permanently or removably, to provide the resultant pad 200.

As shown in FIG. 2C, in one or more embodiments each shoulder pad may include the silicone pad 218 having a bottom portion 220 and a pair of flexible, moveable sidewalls 222, 224. In use, a strap 130 of the counterbalancing apparatus 100 is provided into a channel of the silicone pad 218 so that the strap 130 makes contact with the bottom portion 220 of the silicone pad 218. The moveable sidewalls 222, 224 are moved into position so that the first sidewall 224 covers the strap 130 followed by the second sidewall 222 covering the sidewall 224, thereby securing strap 130 into the channel within the silicone pad 218.

For extra padding and wear comfort, the silicone pad 218 may be provided over the foam pad 214. In one or more embodiments, the foam pad 214 may be a rigid foam material that is used to distribute and carry the weight load of the breasts and weights 300 across the user's shoulders. It should be appreciated that other materials having rigidity and padding capabilities may be used rather than a foam material. The foam pad 214 helps to avoid or reduce the amount or extent that straps 130 and bra strap 410 cut and dig into the wearer's shoulder. The foam pad 214 alone, or in combination with the silicone pad 218, distributes the weight load across the shoulder area. Referring to FIG. 2D, the foam pad 214 may be attached to a soft outer cloth material 210 that contacts the user's skin during use. The outer cloth material 210 is affixed to the foam pad 214 and has a first upper portion 211 and a second upper portion 212 that each wrap up and over the foam pad 214 and/or the silicone pad 218.

Referring to FIGS. 2A-3B, the counterbalancing system of the invention also includes a plurality of weights 300. Each weight 300 has a size and weight able to fit within at least one of the plurality of pockets 160 residing on the body portion 115 of the counterbalancing apparatus 100. During use, the end-user may provide one or more of the plurality of weights 300 into a single one of the pockets 160. Each weight 300 may have a weight ranging from about 0.25 pounds to about 1.0 pound, or even more or less. In one or more embodiments, each weight 300 has a weight of about 0.5 pounds (i.e., ½ pound weight).

In one or more embodiments, each weight 300 may be composed of a flexible iron-infused, non-toxic, plastic material having dimensions suitable to fit within pockets 160. The weights 300 are removable from the pockets 160 for washing, or for transferring to another pocket 160 or a different counterbalance apparatus 100.

The thickness 315 of each weight 300 may range from about 0.10 inches to about 1.0 inch, or even more or less. In a preferred embodiment each weight has a thickness 315 of about 0.5 inches, and even more preferably a thickness 315 of about 0.25 inches (i.e., ¼ inch). While not meant to limit the invention, an exemplary weight 300 may have dimensions of about 3×5 inches with a thickness of about 0.25 inches, and weigh approximately 0.5 pounds (i.e., ½ pound weight). The thickness 315 of 0.25 inches allows for one or more weights 300 to be provided into a single pocket 160 based on end-user needs and preferences at least for counterbalancing breast weight load at the user's bask side. For instance, each pocket 160 may hold at least two (2) of such weights 300.

Depending on the size of the counterbalance apparatus 100 itself (e.g., small, medium, large, extra-large, etc.), the number of pockets 160 across the backside of the body portion will vary based on real estate across the body portion. For instance, a size small may only have enough room to accommodate three (3) pockets, with each pocket capable of holding at least two (2) weights 300. As such, this size small would be able to hold from ½ pound weight (in one pocket only) to about 3 pounds of weight (2 weights in each of the three (3) pockets). As another example, the sizes medium to extra large may have room to accommodate five (5) pockets across the body portion, thereby being able to hold up to 5 pounds of weight (2 weights in each of the five (5) pockets). It should be appreciated that depending upon the size of weight 300, the amount such weight 300 weighs, and the number of pockets 160 across the body portion, the number of weights 300 and amount of pounds added into such pockets will vary depending upon user needs and preferences. That is, for a user with small, light-weighing breasts, such user may only require a few weights 300 in the pockets 160, while a user with large, heavy breasts may need several weights 300 to counterbalance the breast weight load.

In one or more embodiments, the counterbalance apparatus 100 is suitable to work with any type of existing top worn by a user including, but not limited to, a bra, tank top, camisole, shirt, or any other type of top designed and used to hold, secure and lift the breasts. A conventional bra 400 worn by a user 500 is shown in FIGS. 4-6D. The woman's bra 400 has a front cup portion adapted to provide support below the woman's breast (i.e., bust) and a rear or back portion connected by lateral sides that extend around the woman's rib cage to the front cup portion. The counterbalance apparatus 100 may attach to the conventional bra 400 to lift the breast upward and counterbalance the weight load of the breast to the back of the user's body.

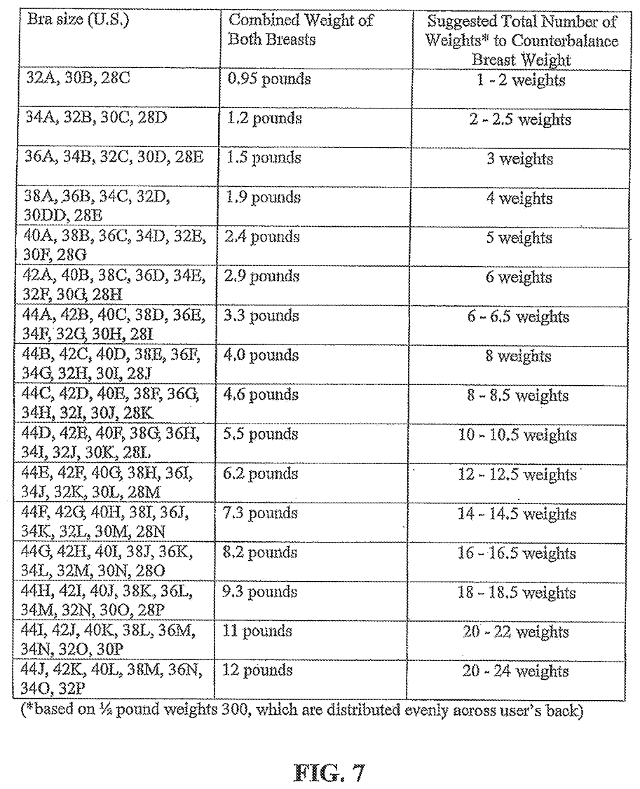

In use, a user of the counterbalance apparatus 100 may first determine how much weight is to be positioned and held within one or more of the plurality of pockets 160 residing on the backside body portion 115. For instance, FIG. 7 illustrates a chart below showing estimated of breast weight of both breasts combined based on volume or size of breast. While FIG. 7 shows estimations of breast weight and suggest weight 300 usage to counterbalance the breast weight load, it should be appreciated that breast weight may vary depending upon breast tissue composition, mass and density. As such, suggested weight 300 usage may also be variable and dependent upon user preferences and needs, which may depend on comfort level, pain relief levels, posture improvement, and the like.

In determining placement of weights 300, in order to balance breast load across to the user's back the user may utilize any number of weights 300 and pockets 160 on the backside body portion 115. As an example, referring to the chart of FIG. 7 for a woman wearing a 32B bra size the estimated combined breast weight is about 1.2 pounds. As such, each breast weighs approximately 0.6 pounds. In order to counterbalance the breast weight load to the woman's back, she may provide a single 0.5 pound weight 300 into each pocket 160 at the backside each in alignment with a respective breast. In doing so, the left breast is counterbalanced with a 0.5 pound weight aligned equidistant from the ground at the left backside of the user, while the right breast is counterbalanced with another 0.5 pound weight aligned equidistant from the ground at the right backside of the user. In one or more preferred embodiments the weights 300 and breasts are positioned and aligned essentially equidistant from the floor with the center of the weights preferably centered to the nipple. In the event the woman determines more weight is needed, she can add another (or as many other) weight(s) 300 into various pockets 160 on the backside body portion 115. For instance, she may add another weight 300 to the center pocket 160 so that she is now carrying and wearing three (3) weights 300.

As another example, for a woman wearing a 34C bra size the estimated combined breast weight is about 1.9 pounds, such that each breast weighs approximately 0.95. This would require a total of four (4) 0.5 pound weights 300 to be positioned and provided into the various pockets 160 at the backside body portion 115. The woman may position and secure two (2) 0.5 pound weights 300 into each pocket 160 that is in alignment with each breast. As such, the woman would have a first pocket 160 having two (2) 0.5 pound weights 300 therein, and a second pocket 160 having another two (2) 0.5 pound weights 300 therein.

In accordance with the invention, a user may use one weight 300 at center pocket 160 that resides substantially at the center point of the user's back, or the user may balance any number of weights across the back. These weights may be balanced at the backside either equally or unequally. For instance, when a woman has had a partial mastectomy less (or more) weight may be needed on one side of the back as compared to the opposing side of such back, thereby resulting in unequal amounts of weights across the woman's back.

It should be appreciated that the weights 300 may be provided within the pockets 160 prior to placing the counterbalance apparatus 100 on the user, or after (e.g., through help of another person). Further, adjustments and/or additions of more weights 300 after wearing apparatus 100 may be implemented in accordance with the invention.

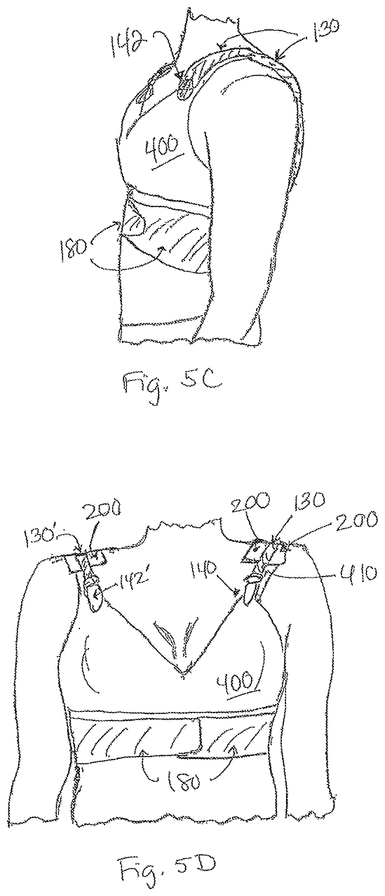

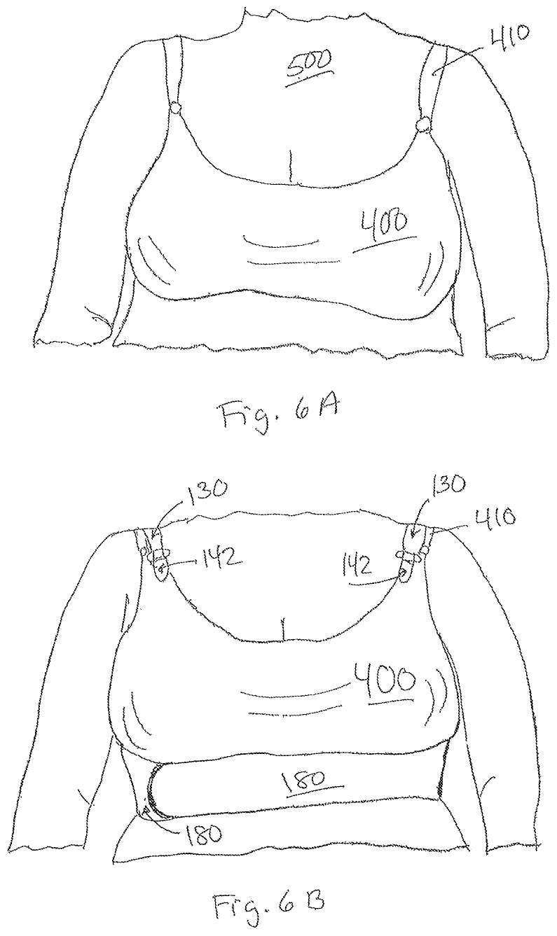

Referring to FIGS. 4-5D, a user 500 attaches a first of the fastening portions 142 of fastener 140 to a first cup portion of the bra 400. The fastening portion 142 may be any suitable fastening mean that securely and removably attaches the apparatus 100 to the bra 400. A first strap 130 connected to this fastening portion 142 is provided over the user's first shoulder and then the counterbalancing apparatus 100 is brought around the user's back, followed by bringing the second strap 130 to the front of the user. The second strap 130′ is securely attached to the second cup portion of bra 400 using the fastening portion 142′ connected at the distal end of strap 130′. It should be appreciated that a variety of different clips and clamps may be implemented to removably fasten the counterbalancing apparatus 100 to the existing bra cup.

Again, the pair of straps 130 are preferably composed of a non-stretch material to provide sufficient and consistent weight counterbalancing across the user's body. The straps may be composed of a variety of different materials that have non-stretch properties for carrying or transferred breast weight across the shoulders and towards the user's back. The straps 130 may also be adjustable for adjusting strap length to accommodate different heights and body measures of users. Once both fastener 140, 140′ are securely attached to the cup portions of the bra 400, the user lifts up both cup portions (preferably simultaneously) thereby lifting both breasts while allowing the body portion 115 retaining the one or more weights 300 to fall at the backside of the user. This allows the weights to fall equidistant (from ground) with the breasts to counterbalance with weight of both the breasts and weights 300 across the user's body. The weights 300 exert an upward force on the bra cup portions pulling the breasts in an upward direction away from the rib cage and diaphragm.

Once the breast weight and weights 300 have been counterbalanced across the front side and the back side of the user, the pair of lateral straps 180 are wrapped around the user's rib cage and are securely fastened to each other at the front of the user's body. Located near the distal ends of each lateral strap 180 are mating connection components 170, 175 that allow for adjustable sizing of the straps 180 around the user's rib cage. Once the straps 180 are secured around the user's torso, the counterbalancing apparatus 100 is affixed to the user's body.

In one or more embodiments, in order to further secure the counterbalancing apparatus 100 to the wearer one or more backside clips or clamps may be used to connect the backside body portion 115 to a backside of the bra 400. These fasteners may removably attach sides of the weighted backside body portion 115 to sides or rear portions of the bra, without substantially constraining the force of the weighted portion on the front cup portion.

In the event the counterbalance weight load places a burden or pain on the user's shoulders, the user may implement the removable shoulder pads 200 as shown in FIG. 5D. These shoulder pads 200 may be positioned under the apparatus 100 straps 130, under the bra straps 410, or under both the apparatus 100 straps 130 and the bra straps 410. The straps may be provided within one or more of the layers of the shoulder pads 200 (e.g., outer cloth material 210, foam pad 214, and/or a silicone pad 218) for securing holding such straps in the pads 200 so that the counterbalance weight load placed on the shoulders is distributed across the shoulder area. The shoulder pads 200 may also be slideable over apparatus 100 straps 130 and/or the bra straps 410 to reduce friction over the woman's shoulders.

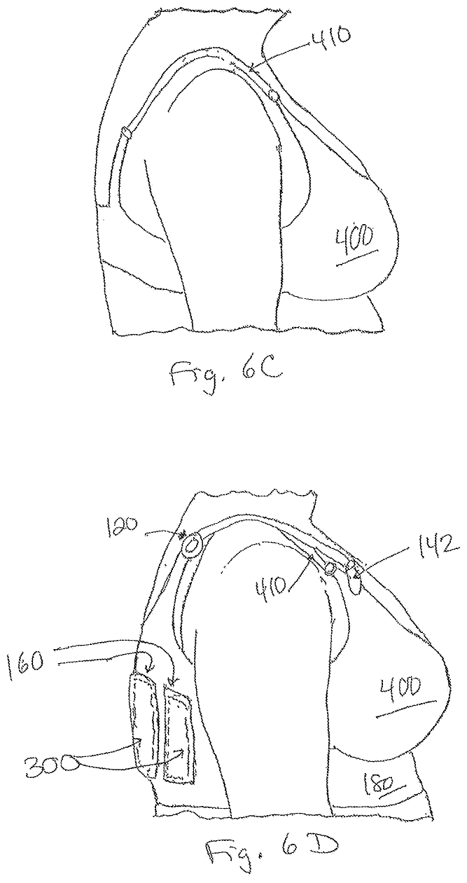

Referring to FIGS. 6A-6D, a user 500 is shown wearing a conventional bra 400. Often due to weight, age, pregnancy, breastfeeding, and the like, a woman's breast will enlarge and/or sag due to gravitational forces on body weight (FIG. 6A). This enlargement and sagging may result in rounding of the woman's shoulders and poor posture (FIG. 6C). Since conventional bras 400 are not designed to deal with breast weight, the weight of the breast is directed at the front of the woman's body causing stress and pain in the neck, shoulders, back, and/or muscle and joint pain. This breast load imbalance through use of a conventional bra also compresses the breast weight on the diaphragm leading to difficulties or problems with breathing.

As shown in FIG. 6B, the present counterbalance apparatus 100 and system raises or pulls the breasts up, thereby lifting breast weight off the diaphragm using one or more weights 300 having equivalent weight measure(s) as the combined breast weight. The weights 300 reside at the back of the wearer of apparatus 100 to counterbalance the breast weight. As such, the counterbalanced weight distribution applies an equal weight force on opposing sides of the wearer to balance weight load (generate a net force of zero).

In use it has been found that the counterbalance apparatus 100 and system of the invention effectively support the breast to balance breast weight load across the skeletal structure or body. As a result, breasts are lifted and stabilized, while the shoulders are pulled back and in a backward down direction to align the spine, upper back and neck into natural positions, thereby correcting or improving posture. The invention reduces, or altogether prevents, rolling of the shoulders and/or slouching.

Not only does the apparatus and systems of the invention counterbalance the weight of breasts and correct posture, it has also been found to lift the breast weight off the shoulders, back, neck and chest, thereby alleviating pain, tension and stress in the shoulder, neck and back areas (see, e.g., FIG. 6D). The counterbalance apparatus 100 has also been found to remove the breast weight off the rib cage and diaphragm allowing the rib cage to feel open and providing for easier breathing due to the lack of compression on the lungs and/or diaphragm. Further, with the breast lifted and posture aligned, the user's stomach appeared to flatten providing the user with a leaner body profile and enabling clothes to fit better and improve self confidence and image.

After wearing the counterbalance apparatus 100 and system of the invention for prolonged times, users found that the weights 300 carried on their backside did not bother them or feel like extra heaviness since the benefits of front to back skeletal weight load counterbalance improved posture and provided an overall feeling of well-being. It was also found that wearing the invention for prolonged times trained the body to recognize correct posture and generated body memory. As such, when the invention was not worn (taken off) the body was naturally brought back into alignment due to such training and body memory provided by the invention.

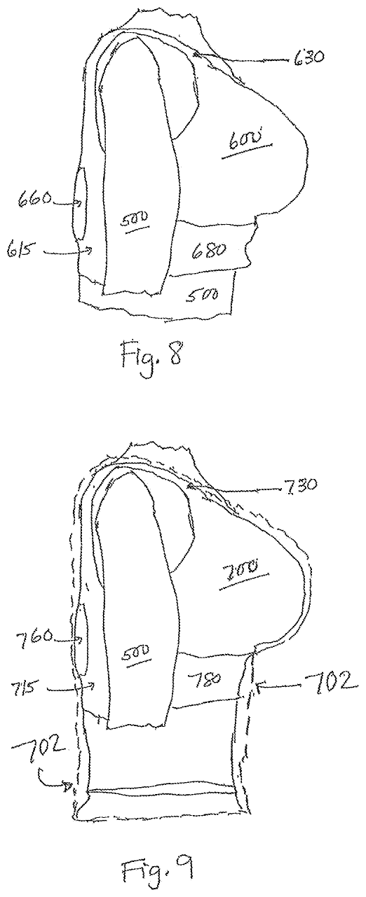

While the present invention has been described in relation to a harness type embodiment, it should be appreciated and understood that the invention is also directed to embodiments where the body portion and straps are components of a bra itself. Referring to FIG. 8, a user 500 is shown wearing a bra 600 of the invention that includes the pair of straps 630 that extend up and over the user's shoulders. The counterbalancing bra 600 of the invention includes a plurality of pockets 660 (as described above) along the back portion 615 thereof, as well as the securing lateral straps 680. One or more weights 300 may be provided in one or more of these pockets 660 to pull up the breast and equilibrate the breast weight load to weights at the back of the user/wearer (which are used interchangeably herein). In still other embodiments in accordance with the invention, the counterbalancing apparatus 700 and system may be incorporated into any type of top 702 worn by a woman/user (e.g., tank top, camisole, shirt, jacket, etc.) The counterbalancing top 700 of the invention may include the pair of straps 730 that extend up and over the user's shoulders, plurality of pockets 760 residing on back portion 715 thereof, and securing lateral straps 780. As with the various other embodiments of the invention, one or more weights 300 may be provided in one or more of these pockets 760 to pull up the breast and equilibrate the breast weight load to weights at the back of the user/wearer (which are used interchangeably herein

While the present invention has been particularly described, in conjunction with a specific preferred embodiment, it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

Thus, having described the invention, what is claimed is:

1. A method for counterbalancing weight retained by bra cups at a front of a bra to a back of a user wearing the bra, the method comprising:
providing a support having a main body portion, a pair of adjustable elongated connector straps and a pair of lateral straps both connected to the main body portion, and a plurality of pockets residing on the main body portion;
providing a plurality of weights;
determining a breast weight of the user's breast retained by the bra cups;
providing a set of weights having a total weight from the plurality of weights, the total weight equals the determined breast weight;
inserting the set of weights into one or more of the plurality of pockets residing on the main body portion;
extending the pair of adjustable elongated connector straps connected to the main body portion up and over the user's shoulders;
connecting the pair of adjustable elongated connector straps to the bra cups of the bra;
pulling the bra cups in an upward direction enabling the main body portion having the set of weights to fall down at the user's back;
extending the pair of lateral straps from the back to the front of the user and securing the pair of lateral straps to each other,
whereby the set of weights pull down on the pair of adjustable elongated connector straps, which in turn, pull up on the bra cups to lift the user's breast up and counterbalance weight to the user's back.

2. The method of claim 1 further including shoulder pads adapted to be positioned on the user's shoulders, the connector straps being slideable over the pads to reduce friction over the user's shoulders.

3. The method of claim 1 wherein the connector straps are non-stretchable when connected to the main body portion.

4. The method of claim 1 further including fasteners connected to a distal end of each of the connector straps, whereby the fasteners connect the connector straps to the bra cups.

5. The method of claim 4 wherein the main body portion is a component of a harness having the fasteners connected at the distal end of the connector straps.

6. The method of claim 1 wherein the support is a bra and the main body portion is a part of the bra.

7. The method of claim 1 wherein the bra is separate from the main body portion and the pair of connector straps.

8. The method of claim 7 wherein the connector straps are slideable over bra straps of the bra to reduce friction over the user's shoulders.

9. The method of claim 7 wherein the main body portion includes fasteners for removably attaching sides of the main body portion to sides or rear portions of the bra without constraining force of the main body portion on the bra cups.

10. The method of claim 1 wherein the support is a wearable tank top having bra cups, and the main body portion is a part of the wearable tank top.

11. The method of claim 1 wherein the breast weight is determined by identifying a bra size on a chart and locating the breast weight corresponding to the bra size on the chart.

12. The method of claim 11 wherein the set of weights comprises a number of weights from the plurality of weights, and is determined using the chart whereby the located breast weight identifies the number of weights needed to counterbalance weight to the user's back.

13. A method for counterbalancing breast weight retained by bra cups at a front of a bra to a back of a user wearing the bra, the method comprising:
providing a harness having a body portion, a pair of adjustable connector straps each having a fastener at an end thereof, and a plurality of pockets residing on the body portion;
providing a plurality of weights;
providing a breast weight of the user's breast retained by the bra cups;
providing a set of weights from the plurality of weights, the set of weights having a combined total weight that equals the breast weight;
inserting the set of weights into one or more of the plurality of pockets residing on the body portion;
positioning the body portion at the user's back and extending the pair of adjustable connector straps up and over the user's shoulders;
connecting the pair of connector straps to the bra cups via the fasteners;
pulling the bra cups in an upward direction enabling the body portion having the set of weights fall down at the user's back;
whereby the set of weights pull down on the pair of adjustable elongated connector straps, which in turn, pull up on the bra cups to lift the user's breast up and counterbalance weight to the user's back.

14. The method of claim 13 wherein the bra is separate from the harness.

15. The method of claim 13 wherein the connector straps are non-stretchable and slideable over bra straps of the bra to reduce friction over the user's shoulders.

16. The method of claim 13 further including shoulder pads adapted to be positioned on the user's shoulders, the connector straps being slideable over the pads to reduce friction over the user's shoulders.

17. The method of claim 13 wherein the body portion includes fastening components for removably attaching sides of the body portion to sides or rear portions of the bra without substantially constraining force of the body portion on the bra cups.

18. The method of claim 13 wherein the harness further includes a pair of lateral straps, the method further including extending the pair of lateral straps from the back to the front of the user and securing the pair of lateral straps to each other.

19. The method of claim 18 wherein the pair of connector straps and the pair of lateral straps are adjustable in size to accommodate different sized body types.

20. The method of claim 13 wherein the set of weights are provided in the plurality of pockets whereby a first half of the weights reside in a first pocket aligned with a first breast, and a second half of the weights reside in a second pocket aligned with a second breast, thereby counterbalancing the breast weight of both breasts to the weights at the user's back to correct posture and lift the breast weight off the shoulders, back, neck and chest of the user.
